(19) United States
(12) Patent Application Publication
(10) Pub. No.: US 2016/0243291 A1
(43) Pub. Date: Aug. 25, 2016

(54) Formulations and Methods for Increasing or Reducing Mucus

(71) Applicant: FORSIGHT VISION5, INC., Menlo Park, CA (US)

(72) Inventors: Cary J. Reich, Los Gatos, CA (US); K. Angela Macfarlane, Menlo Park, CA (US); Yair Alster, Menlo Park, CA (US); Logan Stark, Sunnyvale, CA (US)

(21) Appl. No.: 15/027,573

(22) PCT Filed: Oct. 13, 2014
(86) PCT No.: PCT/US14/60244

§ 371 Date: Apr. 6, 2016

Related U.S. Application Data
(60) Provisional application No. 61/891,270, filed on Oct. 15, 2013.

(51) Int. Cl. A61L 31/12; A61F 9/00; A61L 31/16

(52) U.S. Cl. CPC A61L 31/125; A61L 31/16; A61F 9/0017; A61L 2430/16

(57) ABSTRACT

The present disclosure is directed to a pharmaceutical delivery device useful for the treatment of ocular diseases and disorders through sustained release of therapeutic doses of a pharmaceutical agent, while increasing or reducing formation and/or accumulation of mucus.

### CROSS-REFERENCE TO RELATED APPLICATION

This application claims priority to U.S. Provisional application No. 61/891,270 filed on Oct. 15, 2013, entitled “Formulation to Reduce Mucus”, the disclosure of which is incorporated by reference herein in its entirety.

### FIELD OF THE DISCLOSURE

The present disclosure relates to the controlled, sustained delivery of a pharmaceutical agent of interest to a target tissue of interest, for example, the eye. The present disclosure relates generally to the field of ophthalmics, including ophthalmic devices, for example ocular inserts that deliver a pharmaceutical of interest to the eye while reducing or increasing formation and/or accumulation of mucus.

### BACKGROUND OF THE DISCLOSURE

When an ophthalmic insert containing a pharmaceutical agent is placed on the eye to deliver the pharmaceutical agent in a sustained fashion, the patient may experience side effects related to the ocular insert, for example mucus production/accumulation in the eye. In order to reduce or prevent unwanted or excessive mucus production and/or accumulation attributed to the ocular insert, novel insert composition(s) are needed. The present disclosure addresses these needs.

### SUMMARY OF THE DISCLOSURE

The present disclosure features a composition(s) comprising a polymer matrix and pharmaceutical and/or non-pharmaceutical agents, in which the pharmaceutical and/or non-pharmaceutical agents (e.g., lipids and/or drugs) is dispersed in the polymer matrix.

The present disclosure provides an ocular insert composition of a polymer matrix and one or more excipients, where the excipient reduces production and/or accumulation of mucus in the eye of a subject experiencing and/or at risk of excess mucus formation due to the presence of an ocular insert in the eye. The composition does or does not include one or more pharmaceutical agents.

The present disclosure provides an ocular insert composition of a polymer matrix, optionally including one or more excipients. In one aspect the composition includes one or more excipients, such as colorant or dye, oil, lipids, fatty acids, fatty alcohols, and/or water soluble polymers. In another aspect, the composition does not include an excipient. In one aspect, upon placement of the insert in or on the eye of a subject in need thereof, the insert (with or without one or more excipients) increases production and/or accumulation of mucus in the eye of the subject. In another aspect the ocular insert composition (with or without one or more excipients) includes a pharmaceutical agent. In another aspect the ocular insert composition (with or without one or more excipients) does not include a pharmaceutical agent.

The present disclosure provides an ocular insert composition including a polymer matrix and one or more excipients, but no pharmaceutical agent, where, upon placement of the insert in or on the eye of a subject in need thereof, the excipient reduces production and/or accumulation of mucus in the eye of the subject. The present disclosure provides an ocular insert composition of a polymer matrix, one or more excipients, and one or more pharmaceutical agents, where, upon placement of the insert in or on the eye of a subject in need thereof, the excipient reduces production and/or accumulation of mucus in the eye of the subject. The present disclosure provides an ocular insert composition of a polymer matrix, one or more excipients, and one or more pharmaceutical agents, where, upon placement of the insert in or on the eye of a subject in need thereof, the excipient reduces production and/or accumulation of mucus in the eye of the subject, and the pharmaceutical agent treats a disease or disorder of the eye of the subject. The present disclosure provides an ocular insert composition of a polymer matrix and one or more pharmaceutical agents, but no excipient, where, upon placement of the insert in or on the eye of a subject in need thereof, the pharmaceutical agent treats a disease or disorder of the eye of the subject. When present, the excipient is a colorant or dye, oil, lipids, fatty acids, fatty alcohols, and/or water soluble polymers. The lipid is a phospholipid, e.g., DMPC (1,2-dimyristoyl-sn-glycero-3-phosphocholine). The colorant or dye optionally includes oil, and the colorant is MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7, or any combination(s) thereof. The oil is mineral oil and/or silicone oil. The oil reduces production and/or accumulation of mucus in the eye. The polymer in the polymer matrix is silicone. The silicone is MED-4810, MED-4820, MED-4830, MED-4840, MED-4842, MED-4850, MED1-4855, MED-4860, MED-4870, MED-4880, or any combination(s) of the silicone. The silicone oil is MED-360 and/or MED-370. The water soluble polymer is polyethylene glycol, glycerol, hyalauronic acid, and/or water soluble methylcellulose derivatives.

The present disclosure provides a method of treating a disease or disorder of the eye of a subject in thereof with an ocular insert comprising any one or more compositions of the current disclosure. The present disclosure further provides an ocular insert comprising any one or more compositions of the current disclosure.

The present disclosure provides an ocular insert composition of a polymer matrix, optionally including one or more excipients for treating, preventing (i.e., lowering the risk of), or ameliorating symptoms of dry eye and/or related syndromes in a subject. In one aspect the composition for treating dry eye and/or related syndromes includes one or more excipients, such as colorant or dye, oil, lipids, fatty acids, fatty alcohols, and/or water soluble polymers. In another aspect, the composition for treating dry eye and/or related syndromes does not include an excipient. In one aspect, upon placement of the insert in or on the eye of a subject in need thereof, i.e., suffering from and/or at risk of dry eye and/or related syndromes, the insert (with or without one or more excipients) increases production and/or accumulation of mucus in the eye of the subject. In another aspect the ocular insert composition (with or without one or more excipients) for treating dry eye and/or related syndromes includes a pharmaceutical agent. In another aspect the ocular insert composition (with or without one or more excipients) for treating dry eye and/or related syndromes does not include a pharmaceutical agent.

The present disclosure provides a method of reducing mucus production and/or accumulation in the eye during delivery of a pharmaceutical agent using a delivery device, after the device is inserted onto the eye or inserted into a punctum of the eye. Disclosed herein are observations from pooled safety cohorts of two phase I studies, in which more than two-thirds experienced increased presence of mucus in their eyes when the patients (interchangeably used herein with “subjects”) wore inserts (i.e., drug delivery device of the present disclosure) without pharmaceutical agent as well as inserts with pharmaceutical agents, e.g., bimatoprost.

The device of the current disclosure is, e.g., a ring shaped insert, half-ring shaped insert, flat insert, punctal and intracanalicular occlusion devices including silicone soft plug, collagen punctal or intracanalicular plug, hydrogel soft plug, teflon punctal plug, hydroxyethyl methacrylate (HEM) punctal plug, polycaprolactone (PCL) punctal plug, polydioxanone punctal plug, silicone hydrogel soft plug, and thermosensitive hydrophobic acrylic polymer punctal plug (e.g., SMARTPLUG™, Medennium Inc.), and is for placing or is placed on or in the eye.

The present disclosure also provides a method of treating, ameliorating, and/or reducing mucus formation and/or accumulation on or in an anatomical part of a subject, comprising placement of a pharmaceutical agent delivery device (e.g., an ocular insert) on or in the anatomical part; the device is prepared with a polymerizable or non-polymerizable fluid and comprises an excipient and a pharmaceutical agent; the excipient reduces production and/or accumulation of mucus after the device (e.g., an ocular insert) is placed on or in a target tissue of a subject. The present disclosure provides delaying or reducing accumulation of mucus after the device (e.g., an ocular insert) containing the excipient of the current disclosure is placed on or in a target tissue of a subject.

The present disclosure provides a polymer matrix mixed with the pharmaceutical agent and/or non-pharmaceutical agents (e.g., lipids) and that the polymer matrix comprises a thermosetting polymer that is cured after the pharmaceutical agent and/or non-pharmaceutical agents (e.g., lipids) and the uncured thermosetting polymer are mixed. An example of the thermosetting polymer is silicone, such as MED-4810, MED-4820, MED-4830, MED-4840, MED-4842, MED-4850, MED1-4855, MED-4860, MED-4870, or MED-4880.

The present disclosure provides a composition(s) including a polymer matrix and an excipient, where the excipient reduces or prevents production and/or accumulation of mucus in the eye when the composition(s) is placed on the eye of a subject experiencing and/or at risk of excess mucus formation due to the presence of an ocular insert in the eye. The composition(s) comprises a pharmaceutical agent, and non-pharmaceutical agents (e.g., lipids). Alternatively, the composition(s) does not comprise a pharmaceutical agent, but comprises non-pharmaceutical agents (e.g., lipids). The excipient is a colorant or dye, or component of a colorant or dye, or the excipient is oil. The present disclosure provides colorant or dye included in oil for reducing production and/or accumulation of mucus in the eye of a subject experiencing and/or at risk of excess mucus formation due to the presence of an ocular insert in the eye. The polymer is a silicone, e.g., NuSil products MED-4810, MED-4820, MED-4830, MED-4840, MED-4842, MED-4850, MED1-4855, MED-4860, MED-4870, or MED-4880. The colorant or dye is MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, or MED51-4800-7. The colorant or dye comprising oil is about 0.1-about 20.0% wt. or about 0.5-about 20% wt. of the polymerizable or non-polymerizable fluid. The present disclosure provides composition(s) with oil about 1.0% wt.-about 2.0% wt. of the polymerizable or non-polymerizable fluid. The present disclosure provides composition(s) with color comprising oil or the oil about 0.5% wt.-about 2.0% wt. of the polymerizable or non-polymerizable fluid. The present disclosure provides composition(s) with color comprising oil or the oil about 1.18%/wt. of the polymerizable or non-polymerizable fluid.

The present disclosure provides a formulation or composition(s) of a polymerizable or non-polymerizable fluid for use in a pharmaceutical agent delivery device, including an additive or excipient, and a pharmaceutical agent and/or a non-pharmaceutical agent (e.g., lipid). The additive or excipient in the polymerizable or non-polymerizable fluid reduces production and/or accumulation of mucus after the device is placed on or in a target tissue of a subject. The present disclosure provides composition(s) in which additive or excipient is a colorant or dye, or the excipient is oil. The present disclosure provides composition(s) with a colorant or dye which comprises oil. The present disclosure provides composition(s) with additional excipients including but not limited to: penetration enhancers, such as benzalkonium chloride and/or EDTA, surfactants or co-solvents. The pharmaceutical agent of the present disclosure is in a complex with or in a formulation with cyclodextrin. The present disclosure provides a composition(s) in which a pharmaceutical agent in not in a complex with or not in a formulation with cyclodextrin.

The present disclosure provides composition(s) with a colorant or dye, oil, lipids, fatty acids, fatty alcohols, and/or water soluble polymers in the polymerizable silicone fluid or non-polymerizable fluid about 0.5% to about 20% by weight. The present disclosure provides composition(s) with a colorant or dye, oil, lipids, fatty acids, fatty alcohols, and/or water soluble polymers about 0.5% to about 30% by weight of the composition(s).

The present disclosure provides composition(s) with one or more pharmaceutical agent(s) in which the pharmaceutical agent(s) is about 0.5% to about 30% by weight, about 5% to about 30% by weight, about 5% to about 25% by weight, about 5% to about 22% by weight of the composition(s). The present disclosure provides composition(s) with one or more pharmaceutical agent(s) in which the pharmaceutical agent(s) is about 5%, about 6%, about 7%, about 8%, about 9%, about 10%, about 11%, about 12%, about 13%, about 14%, about 15%, about 16%, about 17%, about 18%, about 19%, about 20%, about 21%, or about 22% by weight of the composition(s).

The present disclosure provides composition(s) with one or more pharmaceutical agent(s) in which the pharmaceutical agent(s) is about 5% to about 25% by weight of the composition(s). The present disclosure provides composition(s) with one or more pharmaceutical agent(s) in which the pharmaceutical agent(s) is about 5% to about 22% by weight of the composition(s). The present disclosure provides composition(s) with one or more pharmaceutical agent(s) in which the pharmaceutical agent(s) is about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition(s). The present disclosure provides composition(s) with one or more pharmaceutical agent(s) in which the pharmaceutical agent(s) is about 7% by weight of the composition(s). The present disclosure provides composition(s) with one or more pharmaceutical agent(s) in which the pharmaceutical agent(s) is about 20% by weight of the composition(s).

The present disclosure provides one or more composition(s) with about 1.18% by weight oil MED-370 and/or MED-360 and about 20% bimatoprost in MED-4830 silicone. The present disclosure provides one or more composition(s) with about 2% MED-4800-1 in MED-4830 silicone.

The one or more composition(s) of this disclosure is configured as a medical device and/or drug product, e.g., an ocular insert, intended to be place on or in the eye. The device has a ring shape. The present disclosure provides a ring shaped ocular insert with a diameter of about 10-about 40 mm or about 20-about 30 mm and a cross-sectional thickness of about 0.1-about 5 mm e.g., about 0.5-about 1.5 mm.

The present disclosure provides a ring shaped ocular insert in which the ring portion of the device has a diameter of about 10-about 40 mm and a cross-sectional thickness of about 0.1-about 5 mm. The present disclosure provides a ring shaped ocular insert with a diameter of about 20-30 mm and the cross-sectional thickness of about 0.5-about 1.5 mm. The current disclosure also provides devices such as half-ring shaped insert, flat insert, punctal and intracanalicular occlusion devices including silicone soft plug, collagen punctal or intracanalicular plug, hydrogel soft plug, teflon punctal plug, hydroxyethyl methacrylate (HEM) punctal plug, polycaprolactone (PCL) punctal plug, polydioxanone punctal plug, silicone hydrogel soft plug, and thermosensitive hydrophobic acrylic polymer punctal plug (e.g., SMARTPLUG™, Medennium Inc.), for inserting or implanting in the eye.

The current disclosure includes a kit comprising a pharmaceutical agent and/or non-pharmaceutical agents (e.g., lipids) delivery device, in which the device is prepared with a polymerizable or non-polymerizable fluid and comprises an excipient and a pharmaceutical agent and/or non-pharmaceutical agents (e.g., lipids), in which the excipient reduces production and/or accumulation of mucus after the device is placed on or in a target tissue of a subject experiencing and/or at risk of excess mucus formation due to the presence of an ocular insert in the eye.

The disclosure features a device comprising any composition(s) described above. The disclosure features a method of using the composition(s) described herein to a treat disease, e.g., alleviate a symptom associated with a disease, or prevent disease progression. The examples of diseases to treat or prevent progression include one or more of: dry eye, symptoms of allergic inflammation of the conjunctiva, postoperative inflammation after cataract surgery, Sjögren's syndrome, corneal abrasion, corneal infection, lid and conjunctival tumors (e.g., basal cell carcinoma), anterior uveitis/iritis, and lower intraocular pressure. The present disclosure also provides a method of improving comfort after a device is inserted or implanted into a body tissue, for example, improving ocular comfort after inserting or implanting the device in the eye.

In certain aspects, the present disclosure provides reducing excess mucus formation in the eye due to a variety of external ocular diseases including keratoconjunctivitis sicca, blepharitis, and allergic conjunctivitis, in subjects experiencing persistence of symptoms of irritation, foreign body sensation, and apparent excessive mucus production, with mild conjunctival inflammation despite appropriate treatment of the underlying disease. In these subjects, the delivery device of the present disclosure comprising a non-pharmaceutical agent, e.g., lipids, but with or without the presence of a pharmaceutically active agent, e.g., bimatoprost, provides comfort and/or reduces mucus formation.

Other features and advantages of the present disclosure will be apparent from the following detailed description and claims.

### BRIEF DESCRIPTION OF THE FIGURES

FIG. 1 shows an embodiment of the ocular device of the present disclosure and its cross-sectional view.

FIG. 2 shows line graphs showing that at 1.18% wt. loading, the oil MED-370 does not significantly affect the release rate of bimatoprost from a MED-4830 based drug delivery device.

FIGS. 3A-B show photographs of a drug delivery device comprising white NuSil colorant. FIG. 3A shows a subject placing a drug delivery device, comprising NuSil colorant, under the lower lid. FIG. 3B is a photograph of the eye after the device is placed onto the eye.

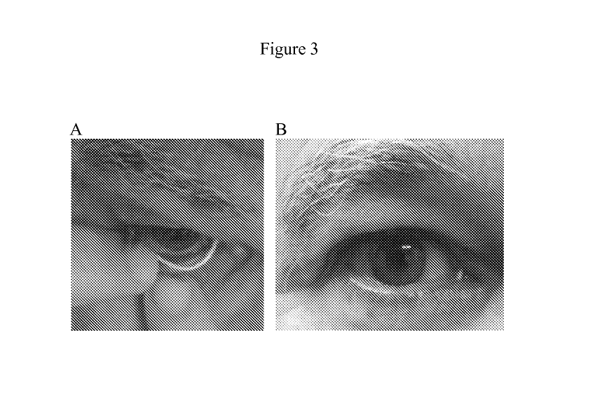

### DETAILED DESCRIPTION OF THE DISCLOSURE

The present disclosure features a composition(s) comprising a polymer matrix and pharmaceutical and/or non-pharmaceutical agents, in which the pharmaceutical and/or non-pharmaceutical agents (e.g., lipids and/or drugs) is dispersed in the polymer matrix.

The present disclosure relates to a pharmaceutical delivery device useful for the treatment of ocular diseases and disorders, including, for example, one or more of macular degeneration, diabetic retinopathy, glaucoma, dry eye, symptoms of allergic inflammation of the conjunctiva, postoperative inflammation after cataract surgery, Sjögren's syndrome, corneal abrasion, corneal infection, lid and conjunctival tumors (e.g., basal cell carcinoma), anterior uveitis/iritis, keratoconus, lazy eye, low vision, ocular hypertension, iris neovascularization and secondary neovascular glaucoma, iridocyclitis, asymmetric cataract, iris atrophy and sluggish reaction to light, general ocular surface disease, and other pathologic conditions such as, e.g., lowering intraocular pressure, through sustained release of therapeutic doses while delaying onset of and/or reducing production and/or accumulation of excessive or unwanted mucus. The present disclosure provides composition(s), methods, and composition(s) for use in the manufacture of a medicament for treating or ameliorating a multifactorial disease in the eye (i.e., dry eye) involving tears and ocular surface that results in symptoms of discomfort, visual disturbance, and tear instability, with the potential to damage the ocular surface, and may be accompanied by increased osmolarity of the tear film and inflammation of the ocular surface. The present disclosure provides a method of reducing or ameliorating mucus production and/or accumulation attributed to the drug delivery device (e.g., an ocular insert) itself, thereby improving comfort level of the inserted or implanted device.

Method of Increasing or Reducing Mucus Formation and/or Accumulation

The present disclosure provides a method of ameliorating and/or reducing mucus formation and/or accumulation comprising placement of a pharmaceutical agent delivery device on or in a target tissue of a subject. The present disclosure further provides a method for increasing mucus formation. The device is prepared with a polymerizable or non-polymerizable fluid and comprises one or more excipient(s) and a pharmaceutical agent and/or non-pharmaceutical agents (e.g., lipids) such that the excipient reduces production and/or accumulation of mucus after the device is placed on or in a target tissue of a subject. The one or more excipient(s) present in the device of the present disclosure delays onset of production and/or accumulation of mucus after the device is placed on or in a target tissue of a subject.

The present disclosure provides an ocular insert composition of a polymer matrix and one or more excipients, where the excipient reduces production and/or accumulation of mucus in the eye of a subject experiencing and/or at risk of excess mucus formation due to the presence of an ocular insert in the eye. The composition does or does not include one or more pharmaceutical agents.

The present disclosure provides an ocular insert composition of a polymer matrix, optionally including one or more excipients. In one aspect the composition includes one or more excipients, such as colorant or dye, oil, lipids, fatty acids, fatty alcohols, and/or water soluble polymers. In another aspect, the composition does not include an excipient. In one aspect, upon placement of the insert in or on the eye of a subject in need thereof, the insert (with or without one or more excipients) increases production and/or accumulation of mucus in the eye of the subject. In another aspect the ocular insert composition (with or without one or more excipients) includes a pharmaceutical agent. In another aspect the ocular insert composition (with or without one or more excipients) does not include a pharmaceutical agent.

The current disclosure provides an ocular insert without (i.e., lacking) an additive or excipient, e.g., a colorant or dye, oil, lipids, fatty acids, fatty alcohols, and/or water soluble polymers, which results in an experience of increased mucus formation and/or accumulation when worn by subjects. Mucus formation and/or accumulation increase when the inserts do not include one or more pharmaceutical agent(s) or drug(s), or when the inserts include one or more pharmaceutical agent(s) or drug(s), e.g., bimatoprost. In certain aspects, the present disclosure provides increasing mucus formation and/or accumulation with inserts including non-pharmaceutical agents (e.g., lipids). The current disclosure provides a drug-delivery device, which reduces or prevents increased or unwanted mucus formation and improves comfort after the device is inserted or implanted in a body tissue, e.g., the eye.

The present disclosure provides an ocular insert composition including a polymer matrix and one or more excipients, but no pharmaceutical agent, where, upon placement of the insert in or on the eye of a subject in need thereof, the excipient reduces production and/or accumulation of mucus in the eye of the subject. The present disclosure provides an ocular insert composition of a polymer matrix, one or more excipients, and one or more pharmaceutical agents, where, upon placement of the insert in or on the eye of a subject in need thereof, the excipient reduces production and/or accumulation of mucus in the eye of the subject. The present disclosure provides an ocular insert composition of a polymer matrix, one or more excipients, and one or more pharmaceutical agents, where, upon placement of the insert in or on the eye of a subject in need thereof, the excipient reduces production and/or accumulation of mucus in the eye of the subject, and the pharmaceutical agent treats a disease or disorder of the eye of the subject. The present disclosure provides an ocular insert composition of a polymer matrix and one or more pharmaceutical agents, but no excipient, where, upon placement of the insert in or on the eye of a subject in need thereof, the pharmaceutical agent treats a disease or disorder of the eye of the subject. When present, the excipient is a colorant or dye, oil, lipids, fatty acids, fatty alcohols, and/or water soluble polymers. The lipid is a phospholipid, e.g., DMPC (1,2-dimyristoyl-sn-glycero-3-phosphocholine). The colorant or dye optionally includes oil, and the colorant is MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7, or any combination(s) thereof. The oil is mineral oil and/or silicone oil. The oil reduces production and/or accumulation of mucus in the eye. The polymer in the polymer matrix is silicone. The silicone is MED-4810, MED-4820, MED-4830, MED-4840, MED-4842, MED-4850, MED1-4855, MED-4860, MED-4870, MED-4880, or any combination(s) of the silicone. The silicone oil is MED-360 and/or MED-370. The water soluble polymer is polyethylene glycol, glycerol, hyalauronic acid, and/or water soluble methylcellulose derivatives.

The present disclosure provides use of a pharmaceutical agent delivery device; the device of the present disclosure is prepared with a polymerizable or non-polymerizable fluid and comprises one or more excipient(s) and one or more pharmaceutical agent(s); the excipient may reduce production and/or accumulation of mucus after the device is placed on or in a target tissue of a subject. The present embodiments also provide a polymerizable or non-polymerizable fluid for use in the manufacture of a medicament for treating a disease or condition in the eye; the polymerizable or non-polymerizable fluid comprises one or more excipient(s) and one or more pharmaceutical agent(s) and the excipient and drug prepared as a drug delivery device; where the one or more excipient(s) delays onset of and/or reduces production and/or accumulation of mucus after the device is placed in a target tissue of a subject.

The excipient or additive for mucus reduction of the present disclosure is a colorant or dye, oil, lipids, fatty acids, fatty alcohols, and/or water soluble polymers. The color included in the composition(s) of the current disclosure includes oil, for example NuSil products: MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7. The oil is about 1.0% wt.-about 2.0% wt. of the polymerizable or non-polymerizable fluid. The color comprising oil or the oil may be about 0.5% wt.-about 2.0% wt. of the polymerizable or non-polymerizable fluid. The color comprising oil or the oil is between about 0.5% wt.-about 2.0%/wt., for example, about 1.18%/wt. of the polymerizable or non-polymerizable fluid. The polymerizable or non-polymerizable fluid of the current disclosure comprise about 0.5% wt.-about 2.0% wt. oil, for example, the about 1.18% wt. oil, for achieving clinically effective mucus reduction. Examples of oil in the color comprising oil of the current disclosure is MED-370 or MED-360 (NuSil Silicone Technology).

One of the methods for the evaluation of mucus level is by using a scale of 0-3 grade levels: 0=No mucus; 0.5=trace mucus; 1=mild mucus; 2=Moderate mucus; and 3=Severe mucus. When a drug delivery device of the present disclosure includes oil, the mucus level in the eye may be at a grade level between 0-1.0; 0.1-1.0, 0.2-1.0, 0.3-1.0, 0.4-1.0, 0.5-1.0, 0.6-1.0, 0.7-1.0, 0.8-1.0, or 0.9-1.0. In some subjects, when the drug delivery device including a colorant or dye, oil, lipids, fatty acids, fatty alcohols, and/or water soluble polymers is placed onto the eye, the mucus level in the eye may be between 1.0-3.0. When a drug delivery device of the present disclosure does not include a colorant or dye, oil, lipids, fatty acids, fatty alcohols, and/or water soluble polymers, the mucus level in the eye may be at a grade level between 0.0-0.5, 1.0-1.1, 1.1-1.2, 1.2-1.3, 1.3-1.4, 1.4-1.5, 1.5-1.6, 1.6-1.7, 1.7-1.8, 1.8-1.9, 1.9-2.0, 2.0-2.1, 2.1-2.2, 2.2-2.3, 2.3-2.4, 2.4-2.5, 2.5-2.6, 2.7-2.8, 2.8-2.9, or 2.9-3.0. Although some subjects have no mucus or trace amount of mucus when a drug delivery device without a colorant or dye, oil, lipids, fatty acids, fatty alcohols, and/or water soluble polymers is placed in or onto the eye of the subjects, in certain aspects of this disclosure, on average (as determined with standard statistical analysis for determining efficacy) subjects wearing device without a colorant or dye, oil, lipids, fatty acids, fatty alcohols, and/or water soluble polymers has higher level of mucus compared to subjects wearing devices with a colorant or dye, oil, lipids, fatty acids, fatty alcohols, and/or water soluble polymers.

The present disclosure provides achieving effective delayed onset of and/or reduction of production and/or accumulation of mucus within from about 10 to about 20 or about 30 or about 40 or about 50 or about 60, or about 90 minutes, about 2 hours (h), about 3 h, about 4 h, about 5 h, about 6 h, about 7 h, about 8 h, about 9 h, about 10 h, about 20 h, about 24 h, about 36 h, about 48 h, or about 76 h, following implantation, insertion, or placement of the delivery device, for example, on or in the eye. The present disclosure provides achieving reduction of mucus in a subject within from about 5 to about 20 or about 30 or about 40 or about 50 or about 60, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h, following implantation, insertion, or placement of the delivery device, for example, on or in the eye. The present disclosure provides achieving effective reduction of mucus in a subject within from about 20 to about 30 or about 40 or about 50 or about 60 minutes, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h following implantation, insertion, or placement of the delivery device, for example, on or in the eye. The present disclosure provides achieving effective reduction of mucus in a subject within about 5 minutes, about 10 minutes, about 15 minutes, about 20 minutes, about 30 minutes, about 40 minutes, about 50 minutes, about 60 minutes, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h following implantation, insertion, or placement of the delivery device, for example, in the eye. The present disclosure provides sustaining mucus reduction in a subject for about 1 week, about 2 weeks, about 3 weeks, about 4 weeks, about 5 weeks, about 2 months, about 3 months, about 4 months, about 5 months, or up to about 6 months or more, following implantation, insertion, or placement of the delivery device, for example, in the eye.

The present disclosure provides achieving effective increase in the production and/or accumulation of mucus within from about 10 to about 20 or about 30 or about 40 or about 50 or about 60, or about 90 minutes, about 2 hours (h), about 3 h, about 4 h, about 5 h, about 6 h, about 7 h, about 8 h, about 9 h, about 10 h, about 20 h, about 24 h, about 36 h, about 48 h, or about 76 h, following implantation, insertion, or placement of the delivery device, for example, on or in the eye. The present disclosure provides achieving increase of mucus in a subject within from about 5 to about 20 or about 30 or about 40 or about 50 or about 60, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h, following implantation, insertion, or placement of the delivery device, for example, on or in the eye. The present disclosure provides achieving effective increase of mucus in a subject within from about 20 to about 30 or about 40 or about 50 or about 60 minutes, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h following implantation, insertion, or placement of the delivery device, for example, on or in the eye. The present disclosure provides achieving effective increase of mucus in a subject within about 5 minutes, about 10 minutes, about 15 minutes, about 20 minutes, about 30 minutes, about 40 minutes, about 50 minutes, about 60 minutes, or about 90 minutes, about 2 h, about 3 h, about 4 h, about 5 h, about 6 h, about 7 h, about 8 h, about 9 h, about 10 h, about 20 h, about 24 h, about 36 h, about 48 h, or about 76 h following implantation, insertion, or placement of the delivery device, for example, in the eye. The present disclosure provides sustaining mucus increase in a subject for about 1 week, about 2 weeks, about 3 weeks, about 4 weeks, about 5 weeks, about 2 months, about 3 months, about 4 months, about 5 months, or up to about 6 months or more, following implantation, insertion, or placement of the delivery device, for example, in the eye.

Composition(s) and Methods for Improving Comfort after Implanting a Drug Delivery-Device

The present disclosure provides composition(s) and methods for improving comfort level in a subject after a drug-delivery device is implanted in or on a target tissue, for example the eye, of the subject. For example, the present disclosure provides improving comfort in the eye; the comfort level is measured using a scale range 0-3, where 0=subject reporting as if nothing in the eye; 0.5=subject is aware of insert but feels normal; 1=subject feels mild discomfort; 2=subject feels moderate discomfort; and 3=subject cannot tolerate the device in the eye. The comfort data can be analyzed using the Likert scale (Likert scaling is a bipolar scaling method, measuring either positive or negative response to a statement), and can be collected at day 1, day 2, day 3, day 4, day 5, day 6, 1 week, 2 weeks, 3 weeks, 4 weeks, 5 weeks, 6 weeks, 7 weeks, 8 weeks, 9 weeks, or 10 weeks. For example, data can be collected for 5-6 days on the relevant week from subjects enrolled in the study and the scores averaged for each subject.

An excipient or an additive in the composition(s) of the drug delivery device of the present disclosure improves comfort by reducing production and/or accumulation of unwanted or excessive mucus (as measured by the scale described in the embodiments of this disclosure). The excipient or additive for mucus reduction of the present disclosure is a colorant or dye, oil, lipids, fatty acids, fatty alcohols, and/or water soluble polymers. The present disclosure provides that a color included in the composition(s) is an oil, for example NuSil products: MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, or MED51-4800-7. The present disclosure provides that oil, lipids, fatty acids, fatty alcohols, and/or water soluble polymers constitutes about 1.0% wt.-about 2.0% wt. of the polymerizable or non-polymerizable fluid. The present disclosure further provides that the color comprising oil or the oil constitutes about 0.5% wt.-about 2.0% wt. of the polymerizable or non-polymerizable fluid. The color comprising oil or the colorant or dye, oil, lipids, fatty acids, fatty alcohols, and/or water soluble polymers of the present disclosure constitutes between about 0.5% wt.-about 2.0%/wt., for example, about 1.18%/wt. of the polymerizable or non-polymerizable fluid. The polymerizable or non-polymerizable fluid of the current disclosure comprises about 0.5% wt.-about 2.0% wt. oil, lipids, fatty acids, fatty alcohols, and/or water soluble polymers, for example, the about 1.18% wt. oil, lipids, fatty acids, fatty alcohols, and/or water soluble polymers, which can be clinically effective in mucus reduction. Examples of oil in the color comprising oil of the current disclosure is MED-370 and/or MED-360 (NuSil Silicone Technology).

The drug-delivery device, for improving comfort in the eye, comprising an excipient or additive, e.g., colorant or dye, oil, lipids, fatty acids, fatty alcohols, and/or water soluble polymers, of the present disclosure is, e.g., a ring shaped insert, half-ring shaped insert, flat insert, or punctal and intracanalicular occlusion device, such as silicone soft plug, collagen punctal or intracanalicular plug, hydrogel soft plug, teflon punctal plug, hydroxyethyl methacrylate (HEM) punctal plug, polycaprolactone (PCL) punctal plug, polydioxanone punctal plug, silicone hydrogel soft plug, or thermosensitive hydrophobic acrylic polymer punctal plug (e.g., SMARTPLUG™, Medennium Inc.).

In certain aspect, the present disclosure relates to affecting psychometric properties of the Ocular Comfort Index (“OCI”) when a subject has a drug delivery device inserted or implanted onto the eye. See, e.g., Johnson et al., Measurement of ocular surface irritation on a linear interval scale with the ocular comfort index, Invest. Ophthalmol. Vis. Sci. (2007), 48(10):4451-8. The OCI exhibits a positive correlation with the Ocular Surface Disease Index (“OSDI”) across a scale of 0-100 (Normal=0-12, Mild=13-22, Moderate=23-32, and Severe=33-100, and a 7 unit change is clinically significant; sub-scale analysis can also be important) (p<0.05-0.0001) and a negative correlation with TBUT (p<0.05-0.0001) and results in improvement in symptoms of dry eye in subjects before and after insertion or implantation of the device. See Johnson.

The present disclosure also provides affecting itching, measured on a scale of 0 to 4, after the drug delivery device is implanted or inserted in the eye. See, e.g., Butrus et al., Comparison of the clinical efficacy and comfort of olopatadine hydrochloride 0.1% ophthalmic solution and nedocromil sodium 2% ophthalmic solution in the human conjunctival allergen challenge model, Clin. Ther. (2000), 22(12):1462-72.

The drug delivery device comprising an excipient or additive, e.g., colorant or dye, oil, lipids, fatty acids, fatty alcohols, and/or water soluble polymers, of the present disclosure is used in treating, preventing (i.e., lowering the risk of), or ameliorating symptoms of dry eye and/or related syndromes in a subject, and/or reducing production and/or accumulation of unwanted and/or excessive mucus.

The present disclosure provides an ocular insert composition of a polymer matrix, optionally including one or more excipients for treating, preventing (i.e., lowering the risk of), or ameliorating symptoms of dry eye and/or related syndromes in a subject. In one aspect the composition for treating dry eye and/or related syndromes includes one or more excipients, such as colorant or dye, oil, lipids, fatty acids, fatty alcohols, and/or water soluble polymers. In another aspect, the composition for treating dry eye and/or related syndromes does not include an excipient. In one aspect, upon placement of the insert in or on the eye of a subject in need thereof, i.e., suffering from and/or at risk of dry eye and/or related syndromes, the insert (with or without one or more excipients) increases production and/or accumulation of mucus in the eye of the subject. In another aspect the ocular insert composition (with or without one or more excipients) for treating dry eye and/or related syndromes includes a pharmaceutical agent. In another aspect the ocular insert composition (with or without one or more excipients) for treating dry eye and/or related syndromes does not include a pharmaceutical agent.

For example, dry eye is measured in a scale of 1-4 for several symptoms using a standard scale in the art. See Behrens et al., Dysfunctional tear syndrome. A Delphi approach to treatment recommendations, Cornea (2006), 25:90-97.

### Excipients for Use in the Delivery Device

The present disclosure provides an ocular insert composition(s) including a polymer matrix, an excipient, and a pharmaceutical agent, where the excipient reduces or prevents (i.e., lowers the risk of) production and/or accumulation of mucus in the eye. For example, the present disclosure provides ocular insert composition(s) in which the excipient is a colorant or dye, oil, lipids, fatty acids, fatty alcohols, and/or water soluble polymers. For example, the present disclosure provides ocular insert composition(s) in which the excipient is a colorant/dye comprising or consisting of an oil.

The excipient present in the ocular insert composition of the present disclosure is a colorant or dye, oil, lipids, fatty acids, fatty alcohols, and/or water soluble polymers. For example, colorant or dye includes oil and the oil reduces production and/or accumulation of mucus in the eye. The colorant comprising oil is, for example, MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7 (available from NuSil Silicone Technology), or combination(s) thereof.

The embodiments of the present disclosure provide a formulation of a polymerizable fluid for use in a pharmaceutical agent delivery device, including an additive or excipient, and a pharmaceutical agent. The additive or excipient in the polymerizable fluid reduces production and/or accumulation of mucus after the device is placed in a target tissue of a subject. When present, the excipient is a colorant or dye, oil, lipids, fatty acids, fatty alcohols, and/or water soluble polymers. The lipid is a phospholipid, e.g., DMPC (1,2-dimyristoyl-sn-glycero-3-phosphocholine). The colorant or dye optionally includes oil, and the colorant is MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7, or any combination(s) thereof. The oil is mineral oil and/or silicone oil. The oil reduces production and/or accumulation of mucus in the eye. The polymer in the polymer matrix is silicone. The silicone is MED-4810, MED-4820, MED-4830, MED-4840, MED-4842, MED-4850, MED1-4855, MED-4860, MED-4870, MED-4880, or any combination(s) of the silicone. The silicone oil is MED-360 and/or MED-370. The water soluble polymer is polyethylene glycol, glycerol, hyalauronic acid, and/or water soluble methylcellulose derivatives.

The oil, lipids, fatty acids, fatty alcohols, and/or water soluble polymers in the polymerizable or non-polymerizable fluid of the present disclosure is, for example, about 0.5% wt. to about 20% wt. of the fluid. The oil, lipids, fatty acids, fatty alcohols, and/or water soluble polymers in the polymerizable or non-polymerizable fluid of the present disclosure is, for example, between about 0.5% wt. to about 30% wt. of the fluid. The pharmaceutical agent of the present disclosure is, for example, about 5% by weight to about 25% by weight of the composition(s). The pharmaceutical agent of the present disclosure is, for example, about 5% by weight to about 22% by weight of the composition(s).

The oil, lipids, fatty acids, fatty alcohols, and/or water soluble polymers used in the composition(s) of the present disclosure is, for example, about 0.5% by weight to 10% by weight of the polymerizable or non-polymerizable fluid. For example, the oil, lipids, fatty acids, fatty alcohols, and/or water soluble polymers is about 0.01%-about 0.5%, about 0.5% -about 0.6%, about 0.6%-about 0.7%, about 0.7%-about 0.8%, about 0.8%-about 0.9%, about 0.9%-about 1.0%, about 1%-about 2%, about 2%-about 3%, about 3%-about 4%, about 4%-about 5%, about 5%-about 6%, about 6%-about 7%, about 7%-about 8%, about 8%-about 9%, or about 9%-about 10% by weight. The oil, lipids, fatty acids, fatty alcohols, and/or water soluble polymers used in the composition(s) of the present disclosure is, for example, about 1.0% wt.-about 2.0% wt. of the polymerizable or non-polymerizable fluid, for example, oil, lipids, fatty acids, fatty alcohols, and/or water soluble polymers is about 1.10% wt., about 1.11% wt., about 1.12% wt., about 1.13% wt., about 1.14% wt., about 1.15% wt., about 1.16% wt., about 1.17% wt., about 1.18% wt., about 1.19% wt., about 1.20% wt., about 1.21% wt., about 1.22% wt., about 1.23% wt., about 1.24% wt., about 1.25% wt., about 1.26% wt., about 1.27% wt., about 1.28% wt., about 1.29% wt., about 1.30% wt., about 1.31% wt., about 1.32% wt., about 1.33% wt., about 1.34% wt., about 1.35% wt., about 1.36% wt., about 1.37% wt., about 1.38% wt., about 1.39% wt., about 1.40% wt., about 1.41% wt., about 1.42% wt., about 1.43% wt., about 1.44% wt., about 1.45% wt., about 1.46% wt., about 1.47% wt., about 1.48% wt., about 1.49% wt., about 1.50% wt., about 1.51% wt., about 1.52% wt., about 1.53% wt., about 1.54% wt., about 1.55% wt., about 1.56% wt., about 1.57% wt., about 1.58% wt., about 1.59% wt., about 1.60% wt., about 1.61% wt., about 1.62% wt., about 1.63% wt., about 1.64% wt., about 1.65% wt., about 1.66% wt., about 1.67% wt., about 1.68% wt., about 1.69% wt., about 1.70% wt., or about 1.71% wt.-about 2.0% wt. of the polymerizable or non-polymerizable fluid.

### Effect of Oil on Mucus Reduction in Additional Anatomical Body Parts

The embodiments of the current disclosure also provide silicone-based sustained release devices such as punctum plug systems and fornix based inserts. The punctum plugs, for drug delivery in various anatomical parts (including the eye) of a subject of the current disclosure are disclosed in, for example, U.S. Pat. No. 7,017,580, issued Mar. 28, 2006, and U.S. publication no. 2005/0232972, published Oct. 20, 2005. Fornix based inserts have been described, see, e.g., Francis, I. C., Aust. J. Ophthalmol. (1984) 12:57-59.

Additional drug delivery devices which may benefit from the methods of reducing mucus during sustained release of drug described herein include Mucoadhesive dosage forms; Ocular Inserts; Collagen shields; Drug presoaked hydrogen type contact lens, for example, OCUFIT® (developed by Escalon Ophthalmics, Inc., Skillman, N.J.), MINIDISC® (Bausch and Lomb, Rochester, N.Y.), and soluble ophthalmic drug insert (SODI®, NODS®, and LACRISERT®).

### Composition(s)/Formulations

The present composition(s) (composition(s) and formulation are used interchangeably throughout the present disclosure) provides for sustained release of a pharmaceutical agent to the eye. The sustained release of a pharmaceutical agent is for a long period (for example up to six months) of time. The composition(s) of this disclosure avoids the therapeutic requirement for frequent administration to maintain a continuous sustained therapeutic level. Further, the composition(s) of the present disclosure lowers the risk of side effects (i.e., although the risk is lowered, individual subject may experience side effects) associated with solution administration such as blurred vision, eyelid redness, permanent darkening of eyelashes, eye discomfort, permanent darkening of iris (to brown), temporary burning sensation during use, growth and/or thickening of the eyelashes, unexpected growth of hair, darkening of the eyelid or of the area beneath the eye.

The present disclosure features a composition(s) comprising a polymer matrix and a pharmaceutical agent, wherein the pharmaceutical agent is dispersed in the polymer matrix.

The pharmaceutical agent for delivery from the delivery device of the present disclosure may comprise, e.g., without being limiting, one or more of the following or their equivalents, derivatives or analogs: thrombin inhibitors; antithrombogenic agents; thrombolytic agents; fibrinolytic agents; vasospasm inhibitors; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); and non-steroidal anti-inflammatories (NSAIDs) (such as salicylate, indomethacin, ibuprofen, diclofenac, flurbiprofen, piroxicam indomethacin, ibuprofen, naxopren, piroxicam and nabumetone). Such anti-inflammatory steroids contemplated for use in the methodology of the embodiments described here, include triamcinolone acetonide (generic name) and corticosteroids that include, for example, triamcinolone, dexamethasone, fluocinolone, cortisone, prednisolone, flumetholone, and derivatives thereof); antiallergenics (such as sodium chromoglycate, antazoline, methapyriline, chlorpheniramine, cetrizine, pyrilamine, prophenpyridamine); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, including antiglaucoma drugs including beta-blockers such as Timolol, betaxolol, levobunolol, atenolol, and prostaglandin analogues such as bimatoprost, travoprost, latanoprost etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and the like.

The delivery device of the present disclosure optionally includes one or more non-pharmacological agents, e.g., lipid, fatty alcohol (e.g., cetyl alcohol, stearyl alcohol), or cyclodextrin. For example, the delivery device includes long chain hydrocarbon oils such as mineral oil and/or may include silicone oils, both polymerizable and non-polymerizable.

The present disclosure features a method of using the composition(s) described herein to treat diseases, e.g., to lower intraocular pressure.

The composition(s) of this disclosure comprises a polymer matrix and a pharmaceutical agent, for example, bimatoprost, where the pharmaceutical agent, for example, bimatoprost, is dispersed in the polymer matrix. The present disclosure provides composition(s) comprising a polymer matrix comprising a thermoplastic polymer or a thermoset polymer, or both.

Examples of thermoplastic polymers include, but are not limited to, acrylonitrile butadiene styrene (ABS), acrylic (PMMA), celluloid, cellulose acetate, cycloolefin copolymer (COC), ethylene-vinyl acetate (EVA), ethylene vinyl alcohol (EVOH), fluoroplastics (PTFE, alongside with FEP, PFA, CTFE, ECTFE, ETFE), ionomers, Kydex, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polycaprolactone (PCL), polychlorotrifluoroethylene (PCTFE), polyethylene terephthalate (PET), polycyclohexylene dimethylene terephthalate (PCT), polycarbonate (PC), polyhydroxyalkanoates (PHAs), polyketone (PK), polyester, polyethylene (PE), polyetheretherketone (PEEK), polyetherketoneketone (PEKK), polyetherimide (PEI), polyethersulfone (PES), polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polytrimethylene terephthalate (PTT), polyurethane (PU), polyvinyl acetate (PVA), polyvinyl chloride (PVC), polyvinylidene chloride (PVDC), and styrene-acrylonitrile (SAN).

The present disclosure provides a composition comprising a polymer matrix comprising a thermosetting polymer. Examples of suitable thermosetting polymers include, but are not limited to, silicones (e.g., MED-4800 series such as MED-4810, MED-4820, MED-4830, MED-4840, MED-4842, MED-4850, MED1-4855, MED-4860, MED-4870, or MED-4880), polyesters (e.g. PET), polyurethanes, vulcanized rubbers, urea-formaldehyde, melamine, epoxy, polyimides, cyanate esters (polycyanurates), vinylesters, bakelite (a phenol-formaldehyde), and duroplast (similar to bakelite).

For example, the polymerizable silicone oil in the composition(s) of the present disclosure is about 0.5% to about 20% by weight of the composition(s). And for example, the pharmaceutical agent is about 5% to about 30% by weight of the composition(s).

For example, the drug delivery device of the present disclosure includes non-polymerizable fluids in the composition(s). Non-polymerizable fluids of the present disclosure include, for example, without being limiting examples, NuSil DDU-310, NuSil MED 400, NuSil MED 360, and mineral oil.

In the composition(s) of this disclosure, the pharmaceutical agent, for example, bimatoprost, for example, is about 0.1% to about 40%, about 1% to about 30%, about 5% to about 30%, about 5% to about 25%, or about 5% to about 22% by weight of the composition(s). In certain instances, the pharmaceutical agent, for example, bimatoprost, for example, is about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition(s). For example, the pharmaceutical agent, e.g., bimatoprost, is about 7% by weight of the composition(s). For example, the pharmaceutical agent, e.g., bimatoprost, is about 20% by weight of the composition(s).

Although it is not intended to be a limitation of the disclosure, it is believed that the pharmaceutical agent, for example, bimatoprost, transports through the silicone matrix to its surface whereupon the agent becomes dispersed, dissolved or otherwise entrained with a body fluid, e.g., tear liquid, blood, or interstitial fluid. The transport may be the result of and/or influenced by diffusion, molecular interaction, domain formation and transport, infusion of body fluid into the matrix or other mechanisms. For delivery to the eye, a therapeutically effective amount of pharmaceutical agent, for example, bimatoprost, transports to the exposed surface of the matrix whereupon tear liquid sweeps away the agent for delivery to target tissue or tissues.

The composition(s) of this disclosure optionally includes a second therapeutic agent. Examples of such optional agents include, but are not limited to, a muscarinic agent, a beta blocker, an alpha agonist, a carbonic anhydrase inhibitor, another prostaglandin analog, an anti-inflammatory agent, an anti-infective agent, a dry eye medication, an anti-scarring agent, an anti-angiogenesis agent, or any combination thereof. The composition(s) of this disclosure includes a second therapeutic agent. Examples of such agents include, but are not limited to, a muscarinic agent, a beta blocker, an alpha agonist, a carbonic anhydrase inhibitor, another prostaglandin analog, an anti-inflammatory agent, an anti-infective agent, a dry eye medication, an anti-scarring agent, an anti-angiogenesis agent, or any combination thereof.

Examples of agents include, but are not limited to, thrombin inhibitors; antithrombogenic agents; thrombolytic agents; fibrinolytic agents; vasospasm inhibitors; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non steroidal anti-inflammatories (NSAIDs) (such as salicylate, indomethacin, ibuprofen, diclofenac, flurbiprofen, piroxicam indomethacin, ibuprofen, naxopren, piroxicam and nabumetone). Examples of such anti-inflammatory steroids contemplated for use with the present lacrimal implants, include triamcinolone acetonide (generic name) and corticosteroids that include, for example, triamcinolone, dexamethasone, fluocinolone, cortisone, prednisolone, flumetholone, and derivatives thereof.); antiallergenics (such as sodium chromoglycate, antazoline, methapyriline, chlorpheniramine, cetrizine, pyrilamine, prophenpyridamine); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil 3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, including antiglaucoma drugs including beta-blockers such as Timolol, betaxolol, levobunolol, atenolol, and prostaglandin analogues such as bimatoprost, travoprost, latanoprost, tafluprost, unoprostone, etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and alpha adrenergic agonists such as brimonidine, clonidine, guanfacine, guanabenz, guanoxabenz, xylazine, tizanidine, methyldopa, fadolmidine, dexmedetomidine, amidephrine, amitraz, anisodamine, apraclonidine, cirazoline, detomidine, dexmedetomidine, epinephrine, ergotamine, etilefrine, indanidine, iofexidine, medetomidine, mephentermine, metaraminol, methoxamine, mivazerol, naphazoline, norepinephrine, norfenefrine, octopamine, oxymetazoline, phenylpropanolamine, rilmenidine, romifidine, synephrine, and talipexole, the like.

The composition(s) of this disclosure also include one or more additives or excipients. For example, it may contain an inert filler material, a salt, a surfactant, a dispersant, a second polymer, a tonicity agent, lipids, or a combination thereof. See, e.g., U.S. Patent Application Publication 2009/0104243.

### Polymer Matrix Drug-Delivery Device

Composition(s) of this disclosure can be prepared as a device, e.g., a medical device, such as an ocular device that can be used to treat eye disease.

The drug-delivery device can comprise one or more drugs or other therapeutic agents, and in some examples, one or more matrix materials to provide sustained release of the drug or other agents. The one or more drugs or other therapeutic agents can migrate from an exposed surface of the drug insert to the target tissue (e.g., ciliary muscles of an eye) based, at least in part, on a solubility of the drugs or agents in the matrix. The rate of migration of the drugs or agents from the exposed surface can also be related to the concentration of drugs or agents dissolved in the matrix. In some examples, the concentration of drugs or agents dissolved in the drug insert can be controlled to provide the desired release rate of the drugs or agents. In addition or in combination, the rate of migration of drugs or agents from the exposed surface can be related to one or more properties of the matrix in which the drugs or agents dissolve, such as the properties of a silicone matrix formulation. In some examples, the drugs or agents included in the drug insert can include liquid, solid, solid gel, solid crystalline, solid amorphous, solid particulate, or dissolved forms. In one such example, solid bimatoprost particles are dispersed in a polymer, e.g., silicone, matrix.

The polymer matrix of the present disclosure is, for example, a thermosetting polymer matrix that is cured after the pharmaceutical agent and the uncured thermosetting polymer are mixed. An example of a thermosetting polymer is silicone, such as MED-4810, MED-4820, MED-4830, MED-4840, MED-4842, MED-4850, MED1-4855, MED-4860, MED-4870, or MED-4880.

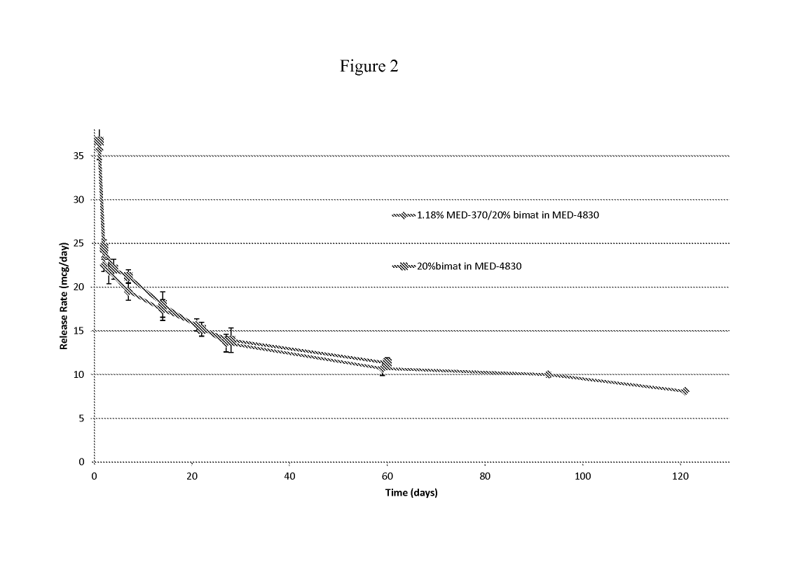

The current disclosure provides that an excipient included in the composition(s), e.g., colorant or dye, oil, lipids, fatty acids, fatty alcohols, and/or water soluble polymers, having a limited or no effect on the drug release rate from a drug delivery device (e.g., ocular insert). Ocular inserts of the present disclosure are prepared, for example, in which NuSil Silicone MED-4830 Parts A and B are mixed with about 20% pharmaceutical agent, e.g., bimatoprost, and about 0.1% wt.-about 20% wt. of color/dye or oil, e.g., about 1.18% wt. MED-370 and/or MED-360 oil. Other ocular inserts of the present disclosure are prepared in which no oil, e.g., MED-370 oil and/or MED-360, is included. For example, about 1.18% wt. MED-370 oil and/or MED-360 is present in the composition(s) without significantly affecting the release rate of the pharmaceutical agent, e.g., bimatoprost, from the ocular insert compared to the composition(s) without oil. See FIG. 2.

The ocular insert composition(s) of the current disclosure is prepared with various oil loadings, as shown in Table 2. For example, the current disclosure provides a composition(s) comprising NuSil MED-4830 prepared with various oils at various loadings, as shown in Table 2. See Example 2. The silicone matrix of the current embodiments is molded to achieve Shoe A hardness of less than the matrix without oil, e.g., between about 10, about 11, about 12, about 13, about 14, about 15, about 16, about 17, about 18, about 19, about 20, about 21, or less than about 25. The current disclosure provides that at least about 20% wt. loading with oil, both polymerizable and non-polymerizable, the silicone MED-4830 is molded for achieving a reasonable Shore A hardness. See Example 3; Qi, et al., Durometer Hardness and the Stress-Strain Behavior of Elastomeric Materials. Rubber Chemistry and Technology (2003), 76(2):419-435.

For example, about 80%-about 85%, about 85%-about 90%, about 90%-about 95%, or about 95%-about 98% of non-polymerizable oil is extracted from the silicone matrix. For example, about 85% (17%/20%) of non-polymerizable oil is extracted from the silicone matrix. For example, about 5%-about 10%, about 10%-about 15%, about 15%-about 20%, about 20%-about 25%, about 25%-about 30%, about 30%-about 35%, or about 35%-40% of polymerizable oil is extracted from the silicone matrix. For example, about 30% or less polymerizable oil, e.g., MED-370 and/or MED-360 oil, is extracted from the silicone matrix. Table 3 provides that the non-polymerizable oils are substantially extracted from the silicone matrix, while the polymerizable oil MED-370 and/or MED-360 is substantially incorporated into the matrix.

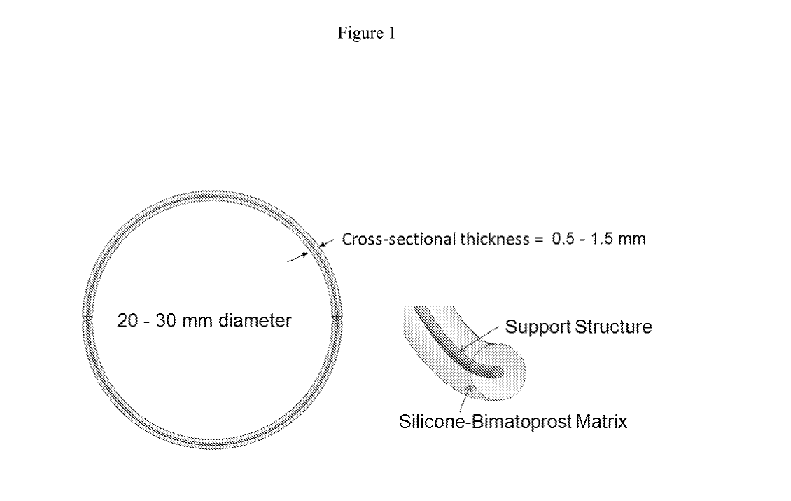

The composition(s) of this disclosure are configured as a device, e.g., a medical device. The medical device is an ocular insert intended to be placed onto the eye. For example, the ocular insert has a ring shape, having a diameter of about 10-about 40 mm or about 20-about 30 mm (e.g., about 20 mm, about 21 mm, about 22 mm, about 23 mm, about 24 mm, about 25 mm, about 26 mm, about 27 mm, about 28 mm, about 29 mm, or about 30 mm) and the cross-sectional thickness can be about 0.1-about 5 mm or about 0.5-about 1.5 mm (e.g., about 0.5 mm, about 0.6 mm, about 0.7 mm, about 0.8 mm, about 0.9 mm, about 1 mm, about 1.1 mm, about 1.2 mm, about 1.3 mm, about 1.4 mm, or about 1.5 mm). FIG. 1 depicts an example of the ring-shaped insert.

The device (e.g., an ocular insert) of the present disclosure is, for example, a silicone device and, for example, has a ring shape, for placing on or in the eye. One silicone ring of the current disclosure includes a colorant and a polymerizable silicone fluid and an Active Pharmaceutical Ingredient (API). The polymerizable silicone fluid delays onset of and/or reduces and/or prevents formation and/or accumulation of mucus in the eye after the drug delivery device (e.g., an ocular insert) is placed in the eye of a subject.

The present disclosure provides a device with a ring within the shaped device having a diameter of about 10 mm-about 40 mm and a cross-sectional thickness of about 0.1 mm-about 5 mm. For example, the ring diameter is about 20 mm-about 30 mm and the cross-sectional thickness is, for example, about 0.5 mm-about 1.5 mm.

The device having a ring shape of the present disclosure is for placing on or in an eye to reduce intraocular pressure. A topical anesthetic can be administered to the site of placement prior to placing the drug delivery device. For example, following optional administration of a drop of anesthetic agent, the eyelids are gently spread open and the ocular insert is placed in the upper and lower fornices. The ocular device of the present disclosure is for keeping in place for a long period of time, for example (up to) between 2 weeks and 6 months, during which time the pharmaceutical agent, for example, bimatoprost, is continuously released onto the eye at a therapeutically effective level.

The pharmaceutical agent, e.g., bimatoprost, thus delivered, is effective for the treatment of diseases or conditions of the eye, e.g., retinopathies, ocular edema and ocular neovascularization. Non-limiting examples of these diseases or conditions include diabetic macular edema, age-related macular degeneration (AMD), cataract, diabetic retinopathy, glaucoma, amblyopia (“lazy eye”), ocular ischemia, uveitis, retinal vein occlusion (central or branch), ocular trauma, surgery induced edema, surgery induced neovascularization, cystoidmacular edema, ocular ischemia, uveitis, and the like.

One non-limiting example of the current disclosure provides continuous release of a pharmaceutical agent, for example, bimatoprost, so as to exert the sustained reduction of intraocular pressure (IOP) or to treat a disease or condition in the eye. Such reduction in IOP can thereby treat or alleviate glaucoma.

Ocular inserts for delivering drug to the eye of a subject of the current disclosure are prepared in various colors, for example, white, skin toned, and pink. The instant embodiments provide white colored inserts without any drug prepared by including about 0.1%-about 5.0% by weight of a white colorant, e.g., MED-4800-1, in a polymer matrix. For example, white colored insert, without any drug may be prepared by including about 0.1-about 0.2%, about 0.2%-about 0.3%, about 0.3%-about 0.4%, about 0.4%-about 0.5%, about 0.5%-about 0.6%, about 0.6%-about 0.7%, about 0.7%-about 0.8%, about 0.8%-about 0.9%, about 0.9%-about 1.0%, about 1.0%-about 1.1%, about 1.1%-about 1.2%, about 1.2%-about 1.3%, about 1.3%-about 1.4%, about 1.4%-about 1.5%, about 1.5%-about 1.6%, about 1.6%-about 1.7%, about 1.7%-about 1.8%, about 1.8%-about 1.9%, about 1.9%-about 2.0%, about 2.0%-about 2.5%, about 2.5%-about 3.0%, about 3.0%-about 3.5%, about 3.5%-about 4.0%, about 4.0%-about 4.5%, or about 4.5%-about 5.0% by weight of a colorant, e.g., MED-4800-1, in a polymer matrix. For example, the current disclosure provides about 2% MED-4800-1 (one of the components of MED-4800-1 is MED-370) included in MED-4830 silicone. The white colored insert may match the color of an insert containing pharmaceutical agent, e.g., bimatoprost.

The instant embodiments provide white colored inserts including a pharmaceutical agent, e.g., bimatoprost, prepared by including about 0.1%-about 5.0% by weight oil and about 1%-about 25% by weight pharmaceutical agent in a polymer matrix. The current disclosure provides about 0.1%-about 0.2%, about 0.2%-about 0.3%, about 0.3%-about 0.4%, about 0.4%-about 0.5%, about 0.5%-about 0.6%, about 0.6%-about 0.7%, about 0.7%-about 0.8%, about 0.8%-about 0.9%, about 0.9%-about 1.0%, about 1.0%-about 1.1%, about 1.1%-about 1.2%, about 1.2%-about 1.3%, about 1.3%-about 1.4%, about 1.4%-about 1.5%, about 1.5%-about 1.6%, about 1.6%-about 1.7%, about 1.7%-about 1.8%, about 1.8%-about 1.9%, about 1.9%-about 2.0%, about 2.0%-about 2.5%, about 2.5%-about 3.0%, about 3.0%-about 3.5%, about 3.5%-about 4.0%, about 4.0%-about 4.5%, or about 4.5%-about 5.0% by weight of an oil and a pharmaceutical agent included in a polymer matrix. The pharmaceutical agent in white colored inserts may be about 1.0%-about 25%, e.g., about 1%-about 2%, about 2%-about 3%, about 3%-about 4%, about 4%-about 5%, about 5%-about 6%, about 6%-about 7%, about 7%-about 8%, about 8%-about 9%, about 9%-about 10%, about 10%-about 11%, about 11%-about 12%, about 12%-about 13%, about 13%-about 14%, about 14%-about 15%, about 15%-about 16%, about 16%-about 17%, about 17%-about 18%, about 18-about 19%, about 19%-about 20%, about 20%-about 21%, about 21%-about 22%, about 22%-about 23%, about 23%-about 24%, or about 24%-about 25% by weight. For example, the current disclosure provides about 1.18% by weight of oil, e.g., MED-370 and/or MED-360, and about 20% by weight of a pharmaceutical agent, e.g., bimatoprost, mixed in a polymer matrix, e.g., MED-4830 silicone.

The embodiments also provide white colored inserts, which include about 0.1%-about 5.0% by weight colorant and about 1.0%-about 25% by weight pharmaceutical agent in a polymer matrix. For example, the current disclosure provides about 0.1%-about 0.2%, about 0.2%-about 0.3%, about 0.3%-about 0.4%, about 0.4%-about 0.5%, about 0.5%-about 0.6%, about 0.6%-about 0.7%, about 0.7%-about 0.8%, about 0.8%-about 0.9%, about 0.9%-about 1.0%, about 1.0%-about 1.1%, about 1.1%-about 1.2%, about 1.2%-about 1.3%, about 1.3%-about 1.4%, about 1.4%-about 1.5%, about 1.5%-about 1.6%, about 1.6%-about 1.7%, about 1.7%-about 1.8%, about 1.8%-about 1.9%, about 1.9%-about 2.0%, about 2.0%-about 2.5%, about 2.5%-about 3.0%, about 3.0%-about 3.5%, about 3.5%-about 4.0%, about 4.0%-about 4.5%, or about 4.5%-about 5.0% by weight of a colorant and a pharmaceutical agent included in a polymer matrix. The pharmaceutical agent in white colored inserts with white colorant is, for example, about 1.0%-about 25%, e.g., about 1.0%-about 2.0%, about 2.0%-about 3.0%, about 3.0%-about 4.0%, about 4.0%-about 5.0%, about 5.0%-about 6.0%, about 6.0%-about 7.0%, about 7.0%-about 8.0%, about 8.0%-about 9.0%, about 9.0%-about 10%, about 10%-11%, about 11%-about 12%, about 12%-about 13%, about 13%-about 14%, about 14%-about 15%, about 15%-about 16%, about 16%-about 17%, about 17%-about 18%, about 18%-about 19%, about 19%-about 20%, about 20%-about 21%, about 21%-about 22%, about 22% about 23%, about 23%-about 24%, or about 24%-about 25% by weight. For example, the current disclosure provides about 2% by weight of a white colorant, e.g., MED-4800-1, and about 20% by weight of a pharmaceutical agent, e.g., bimatoprost, in a polymer matrix, e.g., MED-4830.

The current embodiments also provide an insert that is skin-toned or pink so that the insert blends well with the caruncle/surrounding tissues and is more cosmetically appealing. A pink colored ocular insert of the current embodiments may be developed using a pink or red colorant, e.g., MED-4800-3, which contains about 17.32% by weight Pigment Red 254 and mixing it with oil, e.g., MED-370 oil and/or MED-360. The device color may be chosen by comparing 8 individuals' caruncles and then comparing to the Pantone Formula Guide available from PANTONE®/Graphics. In one embodiment, the representative color may be, e.g., Pantone 698U.

The current embodiments provide evaluation of different loadings of colorant/dye with about 1.0%-about 25% by weight pharmaceutical agent, e.g., bimatoprost, in a polymer matrix. For example, the current disclosure provides different loadings of colorant, e.g., MED-4800-3, mixed with about 20% of a pharmaceutical agent, e.g., bimatoprost, in a polymer matrix, e.g., MED-4830 silicone matrix. For example, about 100 mg-about 1000 mg of a pharmaceutical agent in a polymer matrix is mixed with a colorant and molded for use. For example, present disclosure provides mixing about 806.3 mg of about 20% bimatoprost in MED-4830 matrix with about 0.05 mg of Red color (about 0.006% by weight) in an aluminum dish, and then molding the mixture. The final color can be pinker than Pantone 698U. The current embodiments also provide ocular inserts of colors other than white, skin tone, or shades of pink, e.g., as available for colored contact lenses.

The present disclosure features a device comprising any composition(s) described above.

### Kits

Additional embodiments of the current disclosure include a kit comprising a pharmaceutical agent delivery device, wherein the device is prepared with a polymerizable fluid and comprises an excipient and a pharmaceutical agent and/or non-pharmaceutical agents (e.g., lipids), wherein the excipient reduces production and/or accumulation of mucus after the device is placed on or in a target tissue of a subject.

In some embodiments, the kit comprises a drug delivery device (e.g., ocular insert) comprising a formulation or composition(s) of a polymer matrix, a polymerizable or non-polymerizable fluid, a pharmaceutical agent, and an excipient such as a colorant or dye, oil, lipids, fatty acids, fatty alcohols, and/or water soluble polymers. The pharmaceutical agent in the formulation may be about 1% to about 30%, about 5% to about 30% by weight about 5% to about 25% by weight, about 5% to about 22% by weight of tie composition(s). In some instances, the pharmaceutical agent may be about 5%, about 6° A, about 7%, about 8%, about 9%, about 10%, about 11%, about 12%, about 13%, about 14%, about 15%, about 16%, about 17%, about 18%, about 19%, about 20%, about 21%, or about 22% by weigh of the composition(s). The colorant or dye, oil, lipids, fatty acids, fatty alcohols, and/or water soluble polymers in the formulation may be about 0.1-20% wt. of the polymerizable or non-polymerizable fluid.

### DEFINITIONS

As used herein, the term “bimatoprost” refers to 7-[3,5-dihydroxy-2-(3-hydroxy-5-phenyl-pent-1-enyl)-cyclopentyl]-N-ethyl-hept-5-enamide:

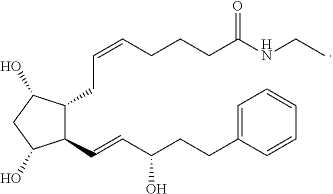 [SMILES: CCNC(=O)CCC/C=C\C[C@@H]1[C@@H](/C=C/[C@@H](O)CCc2ccccc2)[C@H](O)C[C@@H]1O]

Bimatoprost is the API in a product marketed by Allergan as ophthalmic solutions under the trade name LUMIGAN®. It is also the API in a cosmetic formulation known as LATISSE®. The synthesis and purification of bimatoprost is described, e.g., in U.S. Pat. No. 7,157,590.

As used herein, the terms “cure,” “curing,” and “cured” refer to the toughening or hardening of a polymer material by cross-linking of polymer chains, brought about by chemical additives, ultraviolet radiation, electron beam or heat. In one aspect, the polymer is silicone.

As used herein, the term “process,” “processing,” and “processed” refer to reforming intermolecular interactions to remold thermoplastics. Processing is usually achieved by heating and cooling thermoplastics.

As used herein, the term “silicone” refers to polysiloxanes. In one aspect, the silicone has two parts or components, e.g., Part A and Part B, component A or component B. For example, Part A (or component A) may comprise a vinyl containing polydimethylsiloxane and silica (e.g., about 20% silica). Part B (or component B) may comprise of silica (e.g., about 20% silica) and poly(dimethylsiloxane-co-methylhydrosiloxane) (e.g., less than about 3% and where the poly(dimethylsiloxane-co-methylhydrosiloxane) is trimethylsilyl terminated). In another aspect, silicone may be purchased from NuSil Technology or Polymer Systems Technology, Ltd. under a catalog number of the MED-48XX series (e.g. MED-4810, MED-4810 Part A, MED-4810 Part B, MED-4820, MED-4830, MED-4840, MED-4842, MED-4850, MED1-4855, MED-4860, MED-4870, or MED-4880).

As used herein, the term “medical device” refers to a drug-delivery system or device that affects or controls the release and/or delivery of the therapeutic agent in a certain way(s).

As used herein, the terms “ocular insert” and “ocular device” refer to a device, which may or may not contain an API, whose size and shape are designed for ophthalmic application. See, Kumari A. et al., J. Adv. Pharm. Technol. Res. 2010, 1(3): 291-296. In one aspect, the insert may be sterile, thin, multilayered, drug-impregnated, solid or semisolid consistency. In another aspect, the insert may be placed into the cul-de-sac or conjunctival sac. Manufacturing and administration of various ocular inserts have been described in the literature. See, e.g., Kumari et al. In one aspect, the insert or device may be sterile, thin, multilayered, drug-impregnated, solid or semisolid consistency. In another aspect, the insert may be placed into the cul-de-sac or conjunctival sac.

All percentages and ratios used herein, unless otherwise indicated, are by weight.

Although specific reference is made to a ring-shaped ocular insert, medical devices or apparatus having different features can be prepared and used according to the known methods. Such embodiments are within the scope of this disclosure. For example, patent publications US2013/0144128, US2013/0090612, and WO2013/040426, specifically incorporated by reference herein, describe many embodiments of an ocular insert that can be comfortably placed at many locations of the conjunctiva, including along at least a portion of the conjunctival sac. The insert can move when placed on the conjunctiva and can be retained with the eye so as to provide improved comfort for the patient. The insert may comprise a resistance to deflection to retain the insert comfortably within the eye. The insert can be configured in many ways to provide the resistance to deflection. The insert may comprise a matrix comprising the resistance to deflection, and the matrix may comprise a material providing the resistance to deflection. Alternatively or in combination, the insert may comprise a retention structure and a support structure coupled to the retention structure, in which the support structure may contain the therapeutic agent. The retention structure may comprise an inner structure with the support structure comprising the therapeutic agent covering at least a portion of the retention structure, or the retention structure may comprise an outer structure covering at least a portion of the support structure comprising the therapeutic agent.

The insert may be configured such that the insert can be deflected during insertion and removal and may comprise the resistance to deflection for comfort and retention. The insert comprising the resistance to deflection can be comfortably placed at one or more of many locations of the conjunctiva, such that many patients can be treated comfortably and the placement can be adjusted based on the anatomy of the patient and/or physician preference. The one or more locations where the insert can be placed include the inferior conjunctival sac, an inferior temporal location of the conjunctival sac, an inferior nasal location of the conjunctival sac, the superior conjunctival sac, portions of the upper and lower conjunctival sacs near lateral canthus of the palpebral fissure, portions of the upper and lower conjunctival sacs near the medial canthus and caruncle. These areas are well suited to receive structures having relatively large volumes for extended release of one or more therapeutic agents. In one embodiment, the ocular insert is positioned on a region outside an optical zone of an eye.

The insert can be configured in many ways to treat a patient with a pharmaceutical agent, e.g., bimatoprost, for an extended time, and may comprise one or more of a high dose of therapeutic agent, a substantial surface area to release the therapeutic agent, a hoop strength to resist deflection, a bending strength to resist deflection, a shape profile to fit the eye, or a biasing curve to retain the insert, and combinations thereof. The insert may comprise biasing shape so as to retain the insert, for example with a curve, bend, or other deflected shape to retain the insert.

The biasing shape may comprise a resiliently curved biasing spring structure shaped to provide force in response to deflection so as to urge one or more of the first portion or the second portion toward the eye to retain the insert.

In this specification and in the claims that follow, reference is made to a number of terms, which shall be defined to have the following meanings: All percentages, ratios and proportions herein are by weight, unless otherwise specified. All temperatures are in degrees Celsius (° C.) unless otherwise specified.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

A weight percent of a component, unless specifically stated to the contrary, is based on the total weight of the formulation or composition in which the component is included.

By “effective amount” as used herein means “an amount of one or more of the disclosed compounds, effective at dosages and for periods of time necessary to achieve the desired or therapeutic result.” An effective amount may vary according to factors known in the art, such as the disease state, age, sex, and weight of the human or animal being treated. Although particular dosage regimes may be described in examples herein, a person skilled in the art would appreciate that the dosage regime may be altered to provide optimum therapeutic response. For example, several divided doses may be administered daily or the dose may be proportionally reduced as indicated by the exigencies of the therapeutic situation. In addition, the compositions of this disclosure can be administered as frequently as necessary to achieve a therapeutic amount.

“Agent” is used herein to include any other compound that may be contained in or combined with one or more of the disclosed inhibitors that is not a therapeutically or biologically active compound. As such, an agent should be pharmaceutically or biologically acceptable or relevant (for example, an agent should generally be non-toxic to the subject). “Agent” includes a single such compound and is also intended to include a plurality of agents. For the purposes of the present disclosure the term “agent” and “carrier” are used interchangeably throughout the description of the present disclosure and said terms are defined herein as, “ingredients which are used in the practice of formulating a safe and effective pharmaceutical composition.”

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the surface of the eye. Each carrier must be “acceptable” in the sense of being compatible with the other ingredients of the composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, hydroxypropylmethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) gums such as HP-guar; (22) polymers; and (23) other non-toxic compatible substances employed in pharmaceutical formulations.

The term “pharmaceutically acceptable” refers to the fact that the carrier, diluent or agent must be compatible with the other ingredients of the formulation and not deleterious to the recipient thereof.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., dry eye or excessive mucus formation/accumulation). It is understood that this is typically in relation to some standard or expected value, in other words it is relative, but that it is not always necessary for the standard or relative value to be referred to.

The term “treat” or other forms of the word such as “treated” or “treatment” is used herein to mean that administration of a therapeutic agent of the present invention mitigates a disease or a disorder in a host and/or reduces, inhibits, or eliminates a particular characteristic or event associated with a disorder (e.g., dry eye or excessive mucus formation/accumulation).

Insofar as the methods of the present invention are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present invention may occur prior to onset of a disease. The term does not imply that the disease state be completely avoided.

The term “ameliorating a symptom” or other forms of the word such as “ameliorate a symptom” is used herein to mean that administration of a therapeutic agent of the present invention mitigates one or more symptoms of a disease or a disorder in a host and/or reduces, inhibits, or eliminates a particular symptom associated with the disease or disorder prior to and/or post administration of the therapeutic agent.

Throughout the description and claims of this specification the word “comprise” and other forms of the word, such as “comprising” and “comprises,” means including but not limited to, and is not intended to exclude, for example, other additives, components, integers, or steps. As used in the present disclosure, whether in a transitional phrase or in the body of a claim, the terms “comprise(s)” and “comprising” are to be interpreted as having an open-ended meaning That is, the terms are to be interpreted synonymously with the phrases “having at least” or “including at least.” When used in the context of a process the term “comprising” means that the process includes at least the recited steps, but may include additional steps. When used in the context of a molecule, compound, or composition, the term “comprising” means that the compound or composition includes at least the recited features or components, but may also include additional features or components.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if a range of 10 and 15 is disclosed, then 11, 12, 13, and 14 are also disclosed.

In the current disclosure “composition” and “formulation” are used interchangeably and refer to the conventional understanding, as known in the art, of a composition or formulation. “Formulation” as disclosed herein may comprise a solution, suspension, semi-solid, or semi-liquid mixtures of therapeutic agents and/or formulation excipients or formulation agents.

“Solution” according to the current disclosure is a clear, homogeneous liquid form that contains one or more chemical substances dissolved in a solvent or mixture of mutually miscible solvents. A solution is a liquid preparation that contains one or more dissolved chemical substances in a suitable solvent or mixture of mutually miscible solvents. Because molecules of a therapeutic agent substance in solution are uniformly dispersed, the use of solutions as dosage forms generally provides assurance of uniform dosage upon administration and good accuracy when the solution is diluted or otherwise mixed. “Solution” as disclosed herein contemplates any variations based on the current state of the art or variations achieved by one skilled in the art.

“Suspension” according to the current disclosure is a liquid form that contains solid particles dispersed in a liquid vehicle. “Suspension” as disclosed herein contemplates any variations based on the current state of the art or variations achieved by one skilled in the art.

The term “acute” as used herein denotes a condition having a rapid onset, and symptoms that are severe but short in duration. The term “analgesic” as used herein denotes a compound/formulation for the management of intermittent and/or chronic physical discomfort, suitable for long term use. The term “anesthetic” or “anesthesia” as used herein denotes a compound/formulation for the management of acute physical pain, suitable for short term, temporary use, which has an effect that produces numbing or decreased sensitivity in the body part/organ to which the compound/formulation is administered (e.g., decreased corneal sensitivity of the eye). The term “aqueous” typically denotes an aqueous composition wherein the carrier is to an extent of >50%, more preferably >75% and in particular 90% by weight water. The term “chronic” as defined herein is meant a persistent, lasting condition, or one marked by frequent recurrence, preferably a condition that persists/recurs for greater than 3 months, more preferably greater than 6 months, more preferably greater than 12 months, and even more preferably greater than 24 months. The term “comfortable” as used herein refers to a sensation of physical well-being or relief, in contrast to the physical sensation of pain, burning, stinging, itching, irritation, or other symptoms associated with physical discomfort. As used herein the term “symptom” is defined as an indication of disease, illness, injury, or that something is not right in the body. Symptoms are felt or noticed by the individual experiencing the symptom, but may not easily be noticed by others. Others are defined as non-health-care professionals. As used herein the term “sign” is also defined as an indication that something is not right in the body. But signs are defined as things that can be seen by a doctor, nurse, or other health care professional.

The term “more” as used in the present disclosure does not include infinite number of possibilities. The term “more” as used in the present disclosure is used as a skilled person in the art would understand in the context in which it is used.

For the purposes of promoting an understanding of the embodiments described herein, reference made to preferred embodiments and specific language are used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

Unless otherwise defined, all technical and scientific terms used herein have the same meaning as commonly understood by one of ordinary skill in the art to which this disclosure belongs. In the case of conflict, the present specification will control. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed disclosure. In the case of conflict, the present specification, including definitions, will control. In addition, the materials, methods and examples are illustrative only and are not intended to be limiting.

The following examples are illustrative, but not limiting, of the methods and compositions of the present invention. Other suitable modifications and adaptations of the variety of conditions and parameters normally encountered in synthesis and use of the compounds of the present disclosure and that are obvious to those skilled in the art are within the spirit and scope of the present disclosure.

### EXAMPLES

### Example 1

### Mucus Reduction Study

Two Phase I clinical studies have been completed to test the safety of an ocular insert (see, e.g., FIG. 1) for drug delivery. In these clinical studies the ocular insert either with the drug or without the drug was placed in the eye of each subject (i.e., a participant in the study). The cohorts of the two Phase I studies were then pooled to evaluate any adverse effects of the ocular insert. In the pooled “safety” cohorts of two Phase 1 studies (36 patients total), more than two-thirds had increased presence of mucus in their eyes when they wore inserts (i.e., drug delivery device) without drug as well as inserts with drug, e.g., bimatoprost.

In order to investigate means of preventing mucus production, another study was carried out to test effect of oil in mucus production. The oils used in this study are the base oil (MED-370 and/or MED-360) that is part of the formulation of colors available from NuSil Silicone Technology. The colors are: MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, and MED51-4800-7. MED-370-1.18% was tested clinically for mucus reduction. In this study, each subject had two different ocular inserts placed-one insert placed in each of his/her eyes. In one eye, an insert including oil was placed. The ocular insert placed in the other, i.e., the contralateral eye, did not include oil (e.g., control insert).

To evaluate the effect of oil in the device after it is placed in the eye of a subject, subjects were asked to grade their mucus on each day. The Investigator also graded the patients daily. Data in Table 1 is from testing of ocular inserts for 3 days in 5 subjects. Subjects and the Investigator were masked as to which eye had which product. As shown in Table 1, most patients reported lower mucus level (graded as 0-3 grade levels: 0=No mucus; 0.5=trace mucus; 1=mild mucus; 2=Moderate mucus; and 3=Severe mucus) when an insert with oil was placed on the eye compared to the eye with an insert without oil. One patient reported no mucus bilaterally.

Alternative ocular inserts for testing the effect of oil in reducing mucus formation include using non-polymerizable fluids in the composition(s). In such studies, subjects may be provided with ocular inserts that may be prepared with non-polymerizable fluids, such as NuSil DDU-310, NuSil MED 400, or mineral oil, in order to compare whether the oil affects the mucus level when the device made of non-polymerizable fluid is placed in the eye.

### Example 2

### Effect of Oil on Drug Release Rate

A study to determine the effect of oil on the drug release rate was performed in which an insert of the drug delivery device (e.g., an ocular insert) (see FIG. 1) was placed in an in vitro test and compared to a drug delivery device product (e.g., an ocular insert) that was similar except that it did not contain the oil.

Ocular inserts were prepared in which NuSil Silicone MED-4830 Parts A and B were mixed with 20% bimatoprost and about 1.18% wt. MED-370 oil. Other ocular inserts were prepared in the same manner, but without the incorporation of MED-370 oil.

FIG. 2 suggests that at the 1.18% loading, the oil does not significantly affect the release rate of bimatoprost compared to when no oil was present in the formulation.

### Example 3

### Effect of Oil on Silicone Properties

NuSil MED-4830 was prepared with various oils at various loadings, as shown in Table 2. Even at 20% loading with all of the oils used, both polymerizable and non-polymerizable, the silicone MED-4830 could be molded and still had reasonable Shore A hardness.

Table 3 demonstrates that the non-polymerizable oils can be substantially extracted from the silicone matrix, while the polymerizable oil MED-370 is substantially incorporated into the matrix.

Table 4 indicates that elevated mucus was not observed in any eyes at baseline screening, i.e., when subjects did not have any inserts. After washout, when subjects had had inserts for approximately 28 days, most subjects experienced mild mucus. On average, when subjects had bimatoprost-containing inserts, mucus levels trended higher than when they had inserts without drug.

TABLE 1
Clinical Data on Mucus Reduction

| | | Day 1 | Day 2 | Day 3 |
|---|---|---|---|---|
| | | Mucus | | |
| Pt 013 | Insert (no Oil) | 2 | 3 | 2 |
| Pt 013 | Insert (with Oil) | 1 | 2 | 1 |
| Pt 019 | Insert (no Oil) | 2 | 2 | 2 |
| Pt 019 | Insert (with Oil) | 1 | 1 | 1 |
| Pt 025 | Insert (no Oil) | 1 | 1 | 0.5 |
| Pt 025 | Insert (with Oil) | 2 | 2 | 0 |
| Pt 026 | Insert (no Oil) | 0.5 | 2 | 0.5 |
| Pt 026 | Insert (with Oil) | 1 | 1 | 0 |
| Pt 027 | Insert (no Oil) | 0 | 0 | |
| Pt 027 | Insert (with Oil) | 0 | 0 | |

| | Day 1 | Day 2 | Day 3 |
|---|---|---|---|
| | All Data (Patient Data | | |
| | Averaged if both | | |
| | Subject and Investigator | | |
| | Data available) | | |
| Averages: | Mucus | Mucus | Mucus |
| Insert (no Oil) | 1.1 | 1.6 | 1.3 |
| Insert (with Oil) | 1.0 | 1.2 | 0.5 |
| # of Subjects | 5 | 5 | 4 |

Pt: patient

TABLE 2
Properties of NuSil MED 4830 after it has been loaded
with Various Oils

| | Durometer | | |
|---|---|---|---|
| | (Shore A) | Durometer | |
| | after | (Shore A) after | |
| Oil | Molding | One Day | Observations |
| MED-370 - 20% | 11 | 15 | Softer (compared to |
| Vinyl-terminated | | | control) on touch |
| polydimethylsiloxane | | | |
| MED-370 - 1.18% | 33 | 33 | Similar to control |
| loading | | | |
| Mineral Oil - 20% | 15 | 11 | Oil oozes out |
| MED-360 - 20% | 21 | 20 | Similar to Control |
| Polydimethylsiloxane | | | |
| Control (MED-4830 | 30 | 32 | N/A |
| only) | | | |

TABLE 3
Formulations of Silicone Device with or without Oil

| | | | | Fraction of Oil |
|---|---|---|---|---|
| | Silicone | | % Oil | Extracted of |
| | Extractables | Oil Loss | Loss - Si | Total Oil in |
| Oil | (% weight) | (% weight) | Extractables | Formulation |
| MED-370 - | N/A | 10 | 6 | 30% |
| 20% | | | | |
| MED-370 - | | 4.6 | 0.6 | 51% |
| 1.18% | | | | |
| Mineral Oil - | | 21 | 17 | 85% |
| 20% | | | | |
| MED-360 - | | 20 | 16 | 80% |
| 20% | | | | |

| | | | |
|---|---|---|---|
| MED-4830 | 4 | N/A | N/A |
| Only | | | |

TABLE 4
Comparison of No Insert, Insert without Drug, and Prostaglandin-Containing Inserts (“PGA Inserts”) (4.2 mg Bimatoprost)

| | Screening Visit | Post-Washout | | | | | | | |
|---|---|---|---|---|---|---|---|---|---|
| | (Day −28) | Day 0 | Day 1 | Day 7 | Month 1 | Month 2 | Month 3 | Month 4 | Month 5 |
| | n = 31 eyes | n = 31 eyes | n = 31 eyes | n = 31 eyes | n = 29 eyes | n = 29 eyes | n = 24 eyes | n = 20 eyes | n = 8 eyes |
| | Inserts Present in Eyes at Visit Start (OU) | | | | | | | | |
| | | Insert with- | PGA | PGA | PGA | PGA | PGA | PGA | PGA |
| | No Insert | out Drug | Insert | Insert | Insert | Insert | Insert | Insert | Insert |
| | n % | n % | n % | n % | n % | n % | n % | n % | n % |
| None | 31 | 3 | 0 | 1 | 0 | 5 | 2 | 2 | 2 |
| | 100% | 10% | 0% | 3% | 0% | 17% | 8% | 10% | 25% |
| Mild | 0 | 25 | 27 | 22 | 20 | 18 | 16 | 11 | 3 |
| | 0% | 81% | 87% | 71% | 69% | 62% | 67% | 55% | 38% |
| Moderate | 0 | 3 | 2 | 8 | 9 | 6 | 6 | 7 | 3 |
| | 0% | 10% | 6% | 26% | 31% | 21% | 25% | 35% | 38% |
| Severe | 0 | 0 | 2 | 0 | 0 | 0 | 0 | 0 | 0 |
| | 0% | 0% | 6% | 0% | 0% | 0% | 0% | 0% | 0% |

None (0): Normal
Mild (1+): Little mucous identified in one or more areas
Moderate (2+): Substantial amounts of mucous identified in one region
Severe (3+): Substantial amount of mucous identified in a few regions

### Example 4

Ocular inserts for delivering drug to the eye of a subject of the current disclosure were prepared in various colors, for example, white, skin toned, and pink. White colored ocular inserts, without any drug, were prepared by including 2% MED-4800-1 in MED-4830 silicone (one of the components of MED-4800-1 is MED-370). White colored ocular inserts including the drug bimatoprost, were prepared by including 1.18% MED-370 and 20% bimatoprost in MED-4830. In order to test the consistency in releasing drug, a white colored ocular insert including drug was prepared by including 2% MED-4800-1, 20% bimatoprost in MED-4830 (% was measured by weight).

An insert that is skin-toned or pink was developed by evaluating MED-4800-3, which contains 17.32% (by weight), Pigment Red 254 with MED-370 oil. The insert color choice was done by quantitatively analyzing the colors of 8 individuals' caruncles and then comparing to the Pantone Formula Guide available from PANTONE®/Graphics. The representative color chosen was Pantone 698U. Different loadings of MED-4800-3 were evaluated with 20% bimatoprost in MED-4830 matrix. 806.3 mg of 20% bimatoprost in MED-4830 matrix was added to an aluminum dish with 0.05 mg of Red colorant (0.006% by weight). Samples were then molded. The final color was slightly more pink than Pantone 698U. Ocular inserts of colors other than white, skin tone, or shades of pink, e.g., as available for colored contact lenses, are also prepared.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims. It will be understood by those skilled in the art that various changes in form and details may be made therein without departing from the scope of the disclosure encompassed by the appended claims.

What is claimed is:

1. A ring shaped ocular insert for increasing mucus in the eye of a subject, comprising a polymer matrix and an excipient chosen from a colorant or dye, oil, lipid, fatty acid, fatty alcohol, and water soluble polymer.

2. The ring shaped ocular insert of claim 1, further comprising one or more pharmaceutical agents.

3. (canceled)

4. The ring shaped ocular insert of claim 1, wherein the lipid is a phospholipid.

5. The ring shaped ocular insert of claim 4, wherein the phospholipid is DMPC (1,2-dimyristoyl-sn-glycero-3-phosphocholine.)

6. The ring shaped ocular insert of claim 1, wherein the colorant or dye comprises oil, wherein the colorant is selected from the group consisting of MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7, and any combination(s) thereof.

7. The ring shaped ocular insert of claim 1, wherein the oil is chosen from mineral oil and silicone oil.

8. The ring shaped ocular insert of claim 1, wherein the oil increases production and/or accumulation of mucus in the eye.

9. The ring shaped ocular insert of claim 1, wherein the polymer is silicone.

10. The ring shaped ocular insert of claim 9, wherein the silicone comprises MED-4810, MED-4820, MED-4830, MED-4840, MED-4842, MED-4850, MED1-4855, MED-4860, MED-4870, or MED-4880.

11. A ring shaped ocular insert for treating dry eye and/or related syndromes, comprising a polymer matrix comprising an excipient chosen from a colorant or dye, oil, lipid, fatty acid, fatty alcohol, and water soluble polymer.

12. The ring shaped ocular insert of claim 11 further comprising a pharmaceutical agent.

13. The ring shaped ocular insert of claim 11, wherein the composition does not comprise a pharmaceutical agent.

14. (canceled)

15. (canceled)

16. (canceled)

17. The ring shaped ocular insert of claim 11, wherein the colorant is chosen from MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, and MED51-4800-7.

18. The ring shaped ocular insert of claim 11, wherein the lipid is a phospholipid.

19. The ring shaped ocular insert of claim 18, wherein the phospholipid is DMPC (1,2-dimyristoyl-sn-glycero-3-phosphocholine.)

20. The ring shaped ocular insert of claim 11, wherein the oil is chosen from mineral oil and silicone oil.

21. The ring shaped ocular insert of claim 11, wherein the polymer is silicone.

22. The ring shaped ocular insert of claim 7 or 20, wherein silicone oil chosen from MED-360 and MED-370.

23. The ring shaped ocular insert of claim 1 or 11, wherein the water soluble polymer is chosen from polyethylene glycol, glycerol, hyalauronic acid, and water soluble methylcellulose derivatives.

24. (canceled)

25. A method of treating dry eye and/or related syndromes of a subject in need thereof comprising placing an ocular insert into the eye of a subject in thereof, wherein the ocular insert comprises a polymer matrix and an excipient chosen from a colorant or dye, oil, lipid, fatty acid, fatty alcohol, and water soluble polymer.

26. (canceled)

27. The ring shaped ocular insert of claim 1 or 11, wherein the ring has a diameter of about 10-40 mm and a cross-sectional thickness of about 0.1-5 mm.

28. The ring shaped ocular insert of claim 27, wherein the diameter is about 20-30 mm and the cross-sectional thickness is about 0.5-1.5 mm.

29. The ring shaped ocular insert of claim 2 or 12, wherein the colorant or dye, oil, lipid, fatty acid, fatty alcohol, and/or water soluble polymer is about 0.1 to 20% by weight.

30. The ring shaped ocular insert of claim 29, wherein the pharmaceutical agent is about 5% to about 30% by weight of the polymer matrix.

31. A method of increasing mucus formation and/or accumulation in the eye of a subject in need thereof, comprising placement of a ring shaped pharmaceutical agent delivery device, wherein the device is prepared with a polymerizable or non-polymerizable fluid and comprises an excipient and a pharmaceutical agent, wherein the excipient increases production and/or accumulation of mucus after the device is placed in the eye of the subject.

32. (canceled)

33. The ring shaped ocular insert of claim 2 or 12, comprising about 1.18% by weight oil MED-370 and/or MED-360, and about 20% of the pharmaceutical agent in MED-4830 silicone.

34. The ring shaped ocular insert of claim 6 or 17, comprising about 2% MED-4800-1 in MED-4830 silicone.

35. A ring shaped ocular insert for reducing mucus in the eye of a subject comprising a polymer matrix and an excipient chosen from a colorant or dye, oil, lipid, fatty acid, fatty alcohol, and water soluble polymer.

36. The ring shaped ocular insert of claim 35, further comprising one or more pharmaceutical agents.

37. The ring shaped ocular insert of claim 35, wherein the lipid is a phospholipid.

38. The ring shaped ocular insert of claim 37, wherein the phospholipid is DMPC (1,2-dimyristoyl-sn-glycero-3-phosphocholine.)

39. The ring shaped ocular insert of claim 35, wherein the colorant or dye comprises oil, wherein the colorant is selected from the group consisting of MED-4800-1, MED-4800-2, MED-4800-3, MED-4800-4, MED-4800-5, MED-4800-6, MED-4800-7, MED50-4800-1, MED50-4800-2, MED50-4800-3, MED50-4800-4, MED50-4800-5, MED50-4800-6, MED50-4800-7, MED51-4800-7, and any combination(s) thereof.

40. The ring shaped ocular insert of claim 35, wherein the oil is chosen from mineral oil and silicone oil.

41. The ring shaped ocular insert of claim 35, wherein the oil increases production and/or accumulation of mucus in the eye.

42. The ring shaped ocular insert of claim 35, wherein the polymer is silicone.

43. The ring shaped ocular insert of claim 42, wherein the silicone comprises MED-4810, MED-4820, MED-4830, MED-4840, MED-4842, MED-4850, MED1-4855, MED-4860, MED-4870, or MED-4880.

44. The ring shaped ocular insert of claim 40, wherein silicone oil chosen from MED-360 and MED-370.

45. The ring shaped ocular insert of claim 35, wherein the water soluble polymer is chosen from polyethylene glycol, glycerol, hyalauronic acid, and water soluble methylcellulose derivatives.
